(19) United States
(12) Patent Application Publication
(10) Pub. No.: US 2005/0129788 A1
(43) Pub. Date: Jun. 16, 2005

(54) Herbal product to be administered to diabetic individuals and the production method thereof

(76) Inventors: Juan Carlos Agreda Navajas, Vitoria (ES); Fidel Martin Pinto, Vitoria (ES); Efren William Belo Maluendas, Vitoria (ES)

Correspondence Address: DENNIS G. LAPOINTE;LAPOINTE LAW GROUP, PL, PO BOX 1294, TARPON SPRINGS, FL, 34688-1294, US

(21) Appl. No.: 10/512,209

(22) PCT Filed: Apr. 16, 2003
(86) PCT No.: PCT/ES03/00177

(30) Foreign Application Priority Data
Apr. 23, 2002 (ES) P200200943

(51) Int. Cl. A61K 35/78

(52) U.S. Cl. 424/757; 424/769

(57) ABSTRACT

Herbal product, for its administration to diabetic people and process to obtain it. The product is made up of the mixture of two plants, one of them belonging to the Myrtaceae family, preferably of the Syzygium species and the other to the leguminosae-caesalpinoideae family and preferably of the Bauhinia variety. Its production process is developed by the harvesting and separation of the Syzygium seeds and the Bauhinia leaves; their cleaning by pulverizing; their drying till reaching 9% humidity and their grinding, sterilization and homogeneous mixing in the established amounts.

The object of the present invention is a herbal product developed and appropriate for the administration to diabetic people and more specifically to people who suffer from diabetes type II, not dependent from insulin.

The formulation of this herbal product is made up of the mixture of two plants, one of them belonging to the Myrtaceae and the other to the family of the leguminosae-caesalpinoideae. Specifically, the first of them is a Syzygium and the second a Bauhinia.

This product has a hypoglycaemic activity. Its mimetic activity with human insulin allows to reduce the glycose levels in patients with diabetes type II; in an unedited way in pharmacology of herbal formulations.

On the other hand and as they are products obtained from the mixture of two natural plants, the collateral effects on the patients are low or almost nonexistent, which supposes a great advantage as compared to the traditional chemical formulae.

According to the present invention a herbal product is tried to obtain, for its administration to diabetic patients, and more specifically to those suffering from type II, not insulin dependent diabetes.

### Formulation

The qualitative formulation of this product is the one made up of the mixture of two plants, one of them belonging to the Myrtaceae and the other to the family of the leguminosae-caesalpinoideae.

Specifically, the plant belonging to the Myrtaceae is a Syzygium and more specifically in a preferable but not limitative way the species Syzygium cumini or Syzygium jambolanum as selected varieties.

The plant of the leguminosae-caesalpinoideae family is a Bauhinia and specifically in a preferable but not limitative way of the species Bauhinia spp.

The quantitative formulae is determined by a percentage by weight included between 50% and 95% of the Syzygium and a percentage by weight included between 5% and 50% of Bauhinia.

Within these percentages by weight satisfactory results have been reached with a percentage by weight of 85% of Syzygium and a percentage by weight of 15% of Bauhinia.

Particularly samples have been prepared of the product in which the weight of the plants was 450 mg. In this case, the amount of Syzygium is 382.5 mg and the amount of Bauhinia is 67.5 mg.

The presentation of the product can be any of the ones allowed by the pharmacopean techniques but preferably it shall be presented in capsules.

### Production Process

The process is started with the gathering or harvesting of the plants, after which the Syzygium seeds are cleaned as well as the Bauhinia leaves.

Both the seeds of Syzygium and the leaves of Bauhinia are pulverized to 60 mesh and afterwards dried till reaching 9% humidity.

After a quality control process, the grinding phase is started, in which the seeds and the leaves are ground, after which they are sterilized.

In the following phase both ground plants are mixed till reaching a perfect homogenization, after which they are capsulized after passing the corresponding quality controls. Finally the finished product is packed.

### Dosage

As a possible administration dose, satisfactory results have been reached with the following dosage:
A first attacking dose, according to which 4 capsules of 450 mg should be taken, each of them daily for a period of one week.
A maintenance dose of two capsules of 450 mg, each of them daily during the remaining 51 weeks, to complete, between the attacking dose and the maintenance one a period of one year.

It is convenient that the capsule ingestion is carried out twice a day; so that during the attacking dose two capsules shall be taken before breakfast in the morning and two other ones before dinner.

A capsule shall be taken before breakfast and another one before dinner for the maintenance dose.

1. Herbal products for its administration to diabetic people, of the kind suffering from diabetes type II, not insulin dependent, comprising a mixture of two plants,
wherein one of said plants is selected from Myrtaceae family plants of a Syzygium specie and the other of said plants is selected from leguminosae-caesalpinoideae family plants of a Bauhinia specie.

2. Herbal product for its administration to diabetic people according to claim 1,
wherein the plant of the Myrtaceae family is a Syzygium cumini or Syzygium jambolanum, and
wherein the plant belonging to the leguminosae-caesalpinoideae family is a Bauhinia spp.

3. Herbal product for its administration to diabetic people, according to claim 1,
wherein a percentage by weight of the Syzygium specie is about between 50% and 95%; while the percentage by weight of the Bauhinia specie is between about 50% and 5% respectively.

4. Herbal product for its administration to diabetic people according to claim 3, wherein the percentage by weight of the Syzygium specie is about 85% while the percentage of the Bauhinia specie is about 15%.

5. Herbal product for its administration to diabetic people according to claim 1,
wherein the product is in a capsule form containing about 450 mg by weight of both plant so that about 382.5 mg by weight corresponds to a Syzygium cumini and about 67.5 mg by weight corresponds to a Bauhinia spp.

6. Process to obtain a herbal product for its administration to diabetic people, the process comprising:
harvesting of plants selected from Myrtaceae family plants of a Syzygium specie and plants selected from leguminosae-caesalpinoideae family plants of a Bauhinia specie,
separating seeds corresponding to the Syzygium specie and leaves corresponding to the Bauhinia specie;
cleaning by pulverizing of the Syzygium specie seeds and of the Bauhinia specie leaves;
posterior drying the pulverized seeds and leaves until reaching 9% humidity;
grinding the seeds and the leaves;
sterilizing and posteriorly homogeneously mixing the ground seeds and leaves; and
capsulating the sterilized and ground seeds and leaves in capsule form.
